(19) United States
(12) Patent Application Publication
(10) Pub. No.: US 2014/0299739 A1
(43) Pub. Date: Oct. 9, 2014

(54) Personal Magnetic Surgical Instrument System

(71) Applicant: Bradeaux LLC, Peoria Heights, IL (US)

(72) Inventor: Brian P. Bradow, Peoria Heights, IL (US)

(73) Assignee: Bradeaux LLC, Peoria Heights, IL (US)

(21) Appl. No.: 14/244,922

(22) Filed: Apr. 4, 2014

Related U.S. Application Data
(60) Provisional application No. 61/809,459, filed on Apr. 8, 2013.

(51) Int. Cl. A61B 19/02

(52) U.S. Cl. CPC A61B 19/0256; A61B 2019/0257
USPC 248/683; 29/428; 29/458

(57) ABSTRACT

Disclosed is a device to which medical instruments may be removably attached in an operating theater. Such device includes a landing pad attachable to a surgical gown about the region between the abdomen and chest of a person wearing the surgical gown and a magnet attached to the landing pad. Medical instruments are removably attached to the magnet for use by an operator in a surgical theater.

### CROSS-REFERENCE TO RELATED APPLICATIONS

This application claims benefit of provisional application Ser. No. 61/809,459 filed Apr. 8, 2013.

### STATEMENT REGARDING FEDERALLY SPONSORED RESEARCH

Not applicable.

### BACKGROUND

The present disclosure relates generally to accessing certain medical instruments during the course of surgery and more particularly to a unique personal magnetic surgical instrument system therefor.

During standard surgical procedures, access to surgical instrumentation is vitally important. Accessibility to these instruments however, is dependent on multiple factors that are often out of the control of the individual in need of them (referred to here as the “operator”—which may be the surgeon, resident, physician assistant, or any other person participating in the procedure). The experience of the instrument assistant (e.g., scrub technician), the level of case complexity, clutter on the surgical field, the number of people asking for items simultaneously, and distractions within the operating theater (e.g., conversation, music, monitors, phone calls) are only a few such examples. As such, the operator often will maintain increased control over his/her instrument(s), especially if frequent or immediate access is necessary. Many times, the operator simply has a “favorite” instrument that he/she relies on and he/she wants to know exactly where it is at all times. To do this, the operator may simply hold it in his/her hand while simultaneous using another instrument or tuck it in the fold of a surgical drape.

Various devices and methods have been designed to facilitate instrument handling and accessibility. For example, various pouches and straps have been designed, which may be attached to any available sterile surface such as, for example, the drapes or the operators own gown. Instrument receptacles (e.g., magnetic drape) have also been used in an attempt to keep instruments more readily available. Unfortunately, all of these are suboptimal solutions to the operator's needs. The magnetic drape for instance (DEVON™ hands free transfer magnetic drape, Covidien AG) is a stationary item that attempts to prevent lateral movement of instruments that are kept available on the sterile field. However, the instruments are frequently out of the immediate reach of the operator and still are typically passed from the instrument assistant to the operator when they are needed. Such drape also is intended for general use or a shared space, not individualized. The limitations of the surgical instrument storage pack in U.S. Pat. No. 6,308,875, which seeks to make instruments available in a pouch adhered to the operators gown, include that removal of an instrument from the depth of a pouch cannot always be done blindly or safely. The position of an instrument within the pouch also is not fixed, especially when more than one instrument is inside.

U.S. Pat. No. 3,727,658 discloses a receiver for surgical implements which includes a backing sheet of porous foamed elastomeric material on which is placed a plurality of magnetic bars covered by a vacuum formed cover sheet conforming closely to the magnets and adhering to the backing sheet there between. The magnet bars providing raised upper surfaces to provide improved access to surgical implements for removal if desired.

U.S. Pat. No. 3,546,643 discloses a magnetized pad for supporting surgical instruments during an operation comprising a plastic mat in which a number of magnets are embedded, and a sheath of plastic material extending about the surface of the mat.

U.S. Pat. No. 4,826,059 discloses a magnetic tool holder comprising: a generally rectangular main body of flexible material, an apron portion disposed beneath said two upper areas, and at least two sets of a like plurality of magnetic bars.

U.S. Pat. No. 6,530,508 discloses a utility wristband comprising: an elongated flexible band sized in length to wrap around the wrist of a carpenter or like tradesperson, and a plurality of elongated magnetic bars held in spaced substantially parallel transverse relation one to another for holding nails, nuts, bolts, screws, and the like. Such a device clearly would not translate into the surgical setting due to the inherent multiple risks associated with arm mobility (e.g., poor visibility of objects at all times, easily dislodged items, injury to oneself or others, adherence of unwanted objects or instruments, risk of misplacing small items within the patient, etc.

U.S. Published Application No. 2013/0199544 discloses a magnetic drape comprising: a drape body made of a flexible material with magnet units comprising a shielding material in the shape of a cup oriented to have a bottom wall facing toward the undersurface of the drape body.

U.S. Pat. No. 4,944,311 discloses an apparatus for retaining surgical instruments adjacent a surgical field, comprising: a drape having a top surface on which surgical instruments are rested and a bottom surface which lays on a patient and a plurality of instrument retaining magnets secured to said drape.

Despite these proposals, a clear need remains in the art for a surgical instrument storage device that will facilitate instrument control and retrieval by the surgeon or operator, free the surgical assistant from excessive instrument handling tasks, and reduce clutter and confusion in the surgical theater.

It is to solving such drawbacks that the present disclosure is addressed.

### BRIEF SUMMARY

Disclosed is a device to which medical instruments may be removably attached in an operating setting. Such device includes a landing pad attachable to a surgical gown about the region of the mid-chest of a person wearing the surgical gown and a magnet attached to the landing pad. Medical instruments are removably attached to the magnet for use by an operator in a surgical setting.

The corresponding use of the disclosed device is to provide the device and affix one or more medical instruments to the magnet whereby the operator can access the one or more medical instruments in the operating setting.

### BRIEF DESCRIPTION OF THE DRAWINGS

For a fuller understanding of the nature and advantages of the present method and process, reference should be had to the following detailed description taken in connection with the accompanying drawings, in which:

FIG. 1 depicts a prior art green hands free transfer magnetic drape;

FIG. 2 depicts a surgical gown carrying the disclosed personal magnetic surgical instrument system;

FIG. 3 is an image of the components of the disclosed personal magnetic surgical instrument system, viz., landing pad, magnet (e.g., black iron ceramic, neodymium, or similar type), and magnet protective layer;

FIG. 4 is an image of the personal magnetic instrument system of FIG. 3 affixed to a surgical gown and having a surgical clamp (hemostat) held thereto;

FIG. 5 is an image like that of FIG. 4 with the surgeon grasping the surgical clamp (hemostat) from the personal magnetic instrument system for removal of the clamp;

FIG. 6 is an image of a round neodymium magnet suitable for use in the disclosed personal magnetic instrument system;

FIG. 7 is an image of a round neodymium magnet slightly small in diameter than the magnet of FIG. 6 and suitable for use in the disclosed personal magnetic instrument system;

FIG. 8 is an image of an even smaller round neodymium magnet suitable than the magnet of FIG. 7 for use in the disclosed personal magnetic instrument system;

FIG. 9 is an image of a rectangular neodymium magnet suitable for use in the disclosed personal magnetic instrument system;

FIG. 10 is an image of a rectangular neodymium magnet smaller than the magnet of FIG. 9 and suitable for use in the disclosed personal magnetic instrument system;

FIG. 11 is a sectional view taken along line 11-11 of FIG. 8;

FIG. 12 is a vest that carries the disclosed personal magnetic surgical instrument system, such vest being wearable underneath a surgical gown;

FIG. 13 is a sectional view taken along line 13-12 of FIG. 12;

FIG. 14 depicts a vest carrying the disclosed personal magnetic surgical instrument system; and

FIG. 15 depicts the vest of FIG. 13 being worn by the operator under a surgical gown.

The drawings will be described in greater detail below.

### DETAILED DESCRIPTION

The disclosed personal magnetic surgical instrument system is a novel surgical instrument device designed to increase the autonomy of the operator by allowing for safe, immediate, and repetitive access to surgical instrumentation. It is comprised of an optional tactile landing zone material, ideally situated somewhere between the abdomen and the chest of the user or operator with a centrally located magnet(s) upon which a surgical instrument can be temporarily placed.

An additional benefit of the disclosed system is the reduction of misplaced or lost instruments. Also, the need for immediate access to instruments in emergencies or when time is critical to the health of the patient. The landing pad may be omitted in some versions of the disclosed personal magnetic surgical instrument system, as well as the use of multiple landing pads.

A variety of (preferably, non-sharp) common instruments for use with the disclosed personal magnetic surgical instrument system typically will weigh between about 0.7 ounce (19 g) and about 2.7 ounces (76 g). The weights of the instruments that the disclosed personal magnetic surgical instrument system will reliably hold depend upon the strength and number (perhaps, placement) of magnets carried by the personal magnetic surgical instrument system. Typical of those instruments for use with the disclosed personal magnetic surgical instrument system include, for example, Adson forceps, Debakey forceps, Brown forceps, Gerald forceps, Russian forceps, hemostat clamp, mosquito clamp, Kelly clamp, right angle clamp, Freer elevator, Army Navy retractor, metal suction tool, and manual clip applier.

The landing pad may be constructed of a flexible, durable, sterilizable textile material that may be an organic or synthetic fabric, or any other material compatible with the construction and use of the personal magnetic surgical instrument system disclosed herein. Exemplary materials include, for example, woven and non-woven fabrics that optionally may be treated for water resistance or waterproofing. Synthetic textile materials that are heat resistant and otherwise suited for sterilizing and use in the surgical theater include, for example, thermoplastic polymeric yarns, for example nylons, polyesters, PEEK (polyether ether ketone), EVA (ethylene vinyl acetate), silicones, PTFE, FEP, PET/PBT, acetals, PA, PC, and others. Some fabrics include “plastic memory”, which is used to describe textile materials that can be stretched and heated repeatedly and which retain their original shape thereafter (see, e.g., U.S. Pat. No. 4,193,899, issued to Brenner et al.; U.S. Pat. No. 4,820,782, issued to Ueno; and U.S. Pat. No. 4,554,121, issued to Kramer et al., each incorporated herein by reference.

Suitable materials may need to be suitable for gas sterilization using, for example, ethylene oxide (EO) gas, radiation, or steam. Such materials are well known in this art field and can be found, for example in “Zeus Technical Whitepaper Sterilization of Plastics, Zeus Industrial Products, Inc., 2005, the disclosure of which is expressly incorporated herein by reference.

While the magnets may be made of ferrous materials, the weight of such magnets may make them impractical. Lightweight magnets may be useful for use in making the disclosed personal magnetic surgical instrument system. Rare earth metal and ceramic magnets are relatively lightweight. For example, Neodymium magnets are relatively light in weight and are suitable use in present contexts. The diameter of such Neodymium magnets may range from about 1″ to about 2″. Thicknesses of about 1/16″ to about ⅛″ may be suitable for such magnets. It should be recognized, however, that small and larger diameters, as well as thinner and thicker magnets, might find use in present contexts, so long as they suitably retain the surgical instruments of interest and are not too heavy for the user. The shape of the magnets also is not a limitation. Moreover, two or more magnets may be place in a suitable pattern to retain a single instrument.

The disclosed personal magnetic surgical instrument system suitably may be disposable, which is a factor in deciding the materials of construction. The disclosed personal magnetic surgical instrument system also may be reusable, which once again may be a factor in deciding materials of construction.

While non-sharp instruments are preferred, the landing pad may be made from a protective barrier material, such as, for example, Kevlar (an aramide of E. I. du Pont de Nemours and Company, Wilmington, DE), and be sufficiently puncture resistant to protect the operator. Of course, the operator needs to be quite careful in accessing such sharp medical instruments so that a hand or body injury does not occur.

It may be advantageous to coat the magnet with a polymeric material making it somewhat cushioned without sacrificing much, if any, of the strength of the magnetic material. Such coating will prevent the magnet from becoming damaged and may provide better adherence of the instrument(s) to the magnet(s), i.e., non-slip. Suitable sterilizable materials may be used, as described herein.

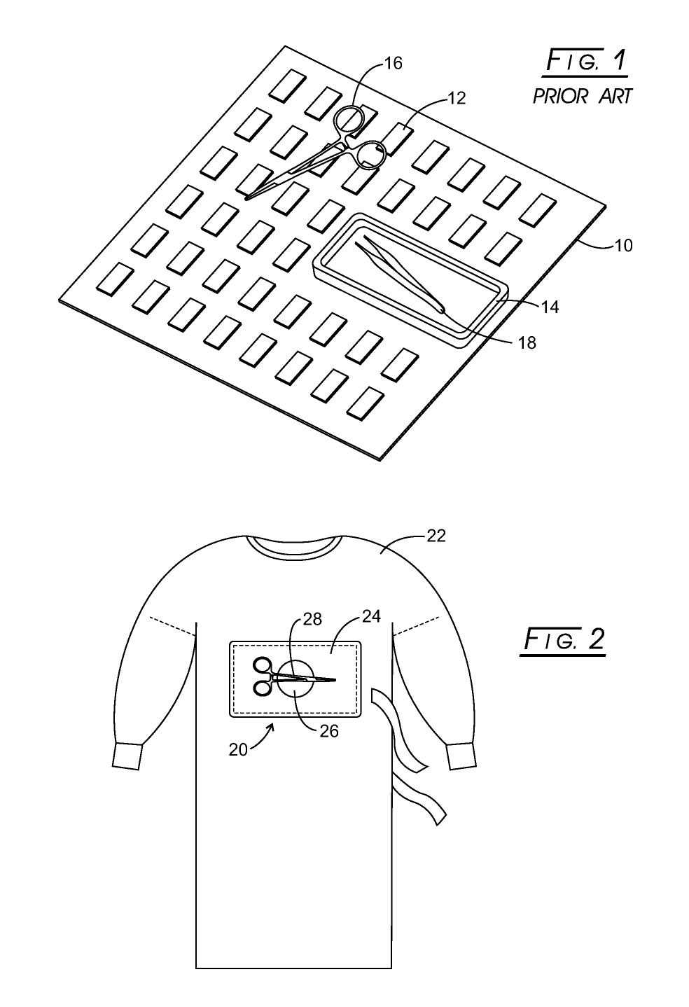

Referring initially to the prior art green hands free transfer magnetic drape of FIG. 1, U.S. Pat. Nos. 3,484,494, 3,546,643, 3,654,047, 3,727,658, and 4,944,311; and U.S. Publications Nos. 2000/0485262004/0118410, and 2013/0199544, each depict a variation of such magnetic drapes for surgical instruments, which the disclosed personal magnetic surgical instrument system improves upon. In particular, a planar substrate, 10, retains a plurality of magnets, such as typified by a magnet, 12, and a tray, 14. Magnet 12 and other similar magnets retain a surgical clamp, 16, while tray 14, retains pickups, 18. Planar substrate 10 is placed on the patient in the operating room (“OR”) with a variety of surgical instruments placed thereon for use by the operator during surgery. The limitations on such a system have been noted above.

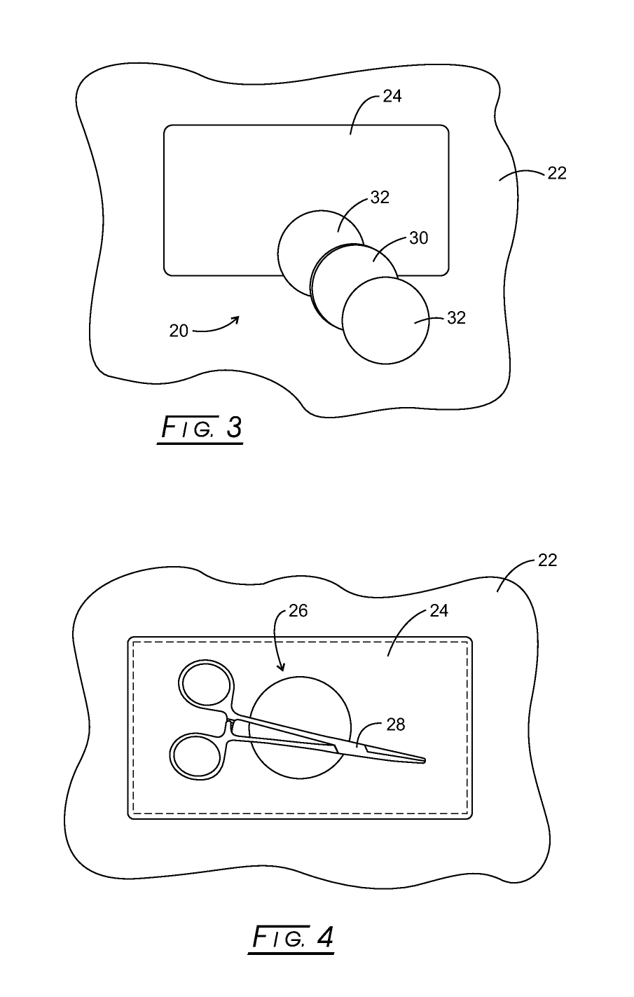

FIG. 2 reveals the disclosed personal magnetic instrument system, 20, affixed to a surgical gown, 22, as worn by an operator, typically a surgeon, about the chest to abdomen area. Personal magnetic instrument system 20 may affixed either to the inside or the outside of the surgical gown, or to the operator's clothing (e.g., shirt, scrubs, etc.) either as an original article of manufacture or as an after-market added product. As noted above, personal magnetic instrument system 20 in FIG. 2 consists of a landing pad, 24, and magnet assembly, 26, retaining a surgical clamp 28. Referring to FIGS. 3 and 4, magnet assembly 26 is seen to include a magnet, 30, and protective barriers, 32, making a sandwich construction for cushioning the magnet. While magnet 30 is shown round in shape, other shapes may be used to advantage as discussed above. Protective barriers 32 protect the magnet and may be made of Kevlar or other protective material. Barriers 32 need be only sufficiently thick to provide a degree of protection to magnet 30 which may be made of ceramic or other fragile material. Typically magnet 30 and protective barriers 32 are affixed by adhesive approved for such medical use; although, the means of attachment is not limitative on the disclosed personal magnetic instrument system.

In FIG. 5, gown 22 is shown worn by an operator. The hand of the operator, 34 is grasping surgical clamp 28 being carrier by personal magnetic instrument system 20. Muscle memory of the operator will enable the operator to remove and replace surgical clamp 28 or other surgical instrument without having to look down at the instrument, making the disclosed personal magnetic instrument system easy and quick to use by the operator, even when a plurality of medical instruments are accessible by the operator.

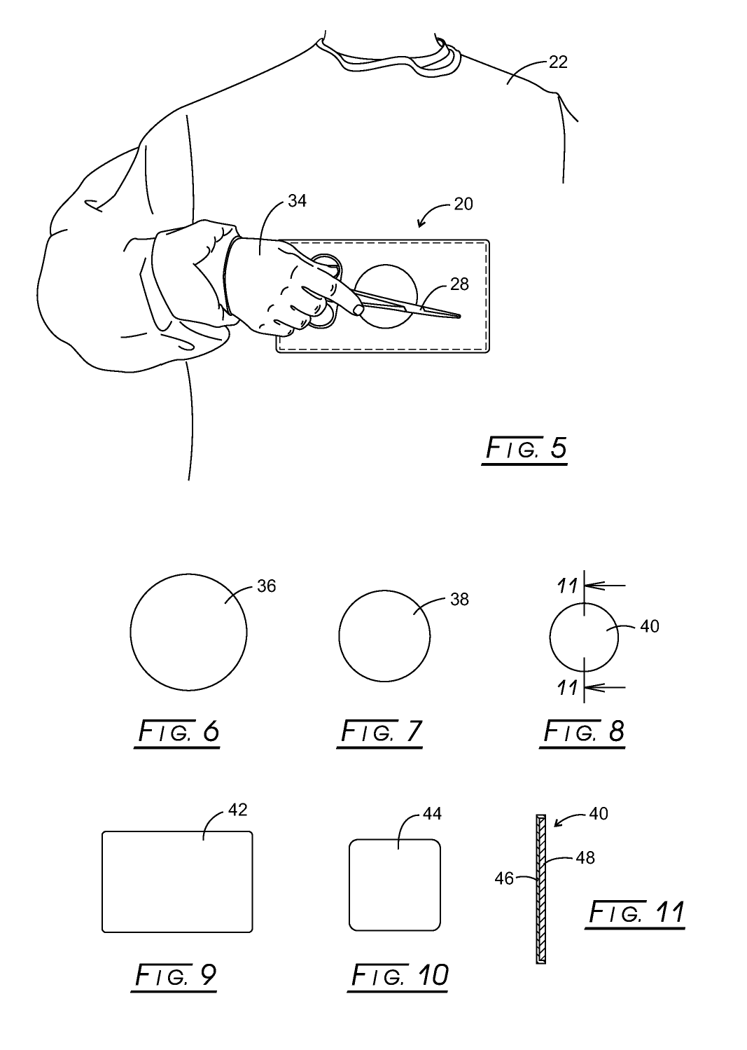

Referring now to FIGS. 6-11, various shaped and size magnet assemblies are illustrated. In particular, three different size round magnet assemblies, 36, 38, and 40, are shown in FIGS. 6-8, respectively. The magnets desirably will be relatively light in weight, making ceramic and rare earth magnetics desirable for use in the disclosed personal magnetic instrument system. Thicknesses will be such that the magnetic strength of the one or more magnets design to retain each particular medical instrument will make their retention certain, yet permit facile removal by the operator. Typical thicknesses of rare earth magnetics can range from about 1/16″ to about ⅛″ in thickness. As discussed above, desirably such magnets will have a thick protective barrier atop them or sandwiched around them.

Two different size rectangular magnet assemblies, 42 and 44, are shown in FIGS. 9 and 10, respectively. The corners of the magnets have been rounded to prevent sharp corners from causing injury or damage. Additionally, the magnets may be chamfered for additional safety. In FIG. 11, magnet assembly 40 is seen to be composed of a magnet, 46, and protective barrier, 48.

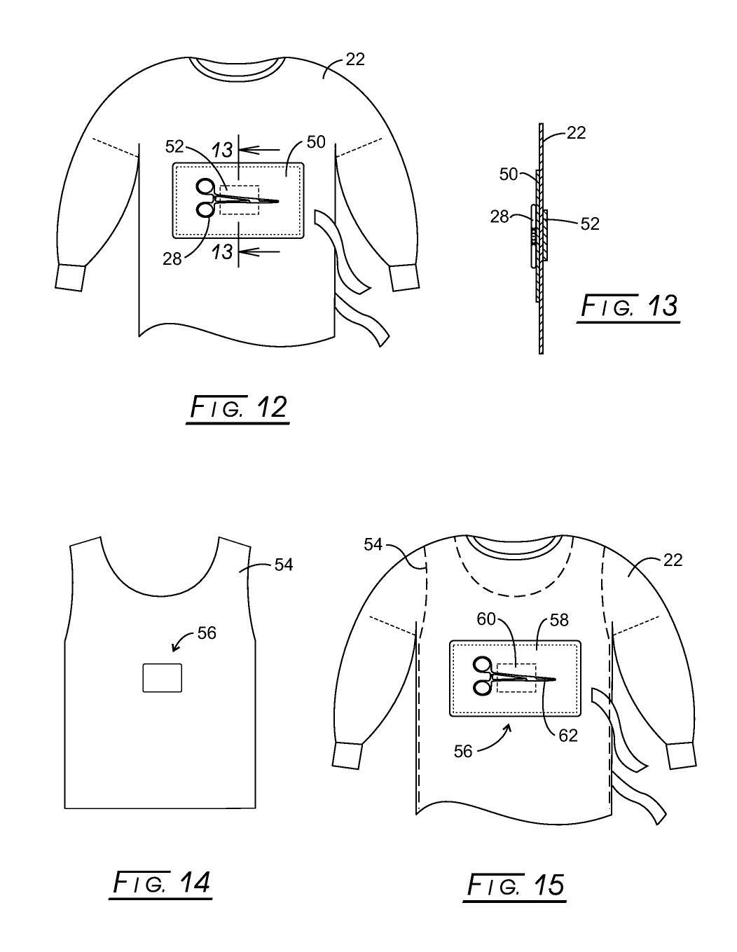

Referring now to FIG. 12, surgical gown 22 carries a landing pad, 50, on the outside of gown 22. On the inside of surgical gown 22 is a magnet, 52, shown in phantom. FIG. 13 illustrates such assembly in cross-section. The location of magnet 52 can be outlined on landing pad 50 for providing a visual cue to the operator. This also can be done for multiple magnets carried on the inside of the surgical gown.

FIG. 14 illustrates a vest, 54 carrying a personal magnetic instrument system, 56, which again can be located on either the inside or the outside; although, the outside is appropriate for this embodiment. As illustrated in FIG. 15, the operator wears vest 54, shown in phantom, underneath surgical gown 22. Vest 54 carries personal magnetic instrument system 56 (see FIG. 14), consisting of a landing pad, 58, a magnet, 60 (shown in phantom) carrying a surgical clamp, 62. Of course, the strength of the magnet may need to be slightly stronger in this embodiment. Sterilization procedures may be obviated or lessened by this embodiment.

While the device and method have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

I claim:

1. A device to which medical instruments may be removably attached in an operating theater, which comprises:
(a) a landing pad attachable to a surgical gown about the region between the abdomen and chest of a person wearing the surgical gown; and
(b) a magnet attached to the landing pad,
whereby medical instruments can be removably attached to the magnet.

2. The device of claim 1, wherein the magnet is coated with a polymeric material.

3. The device of claim 1, wherein more than one magnet is attached to the landing pad.

4. The device of claim 1, wherein the landing pad is woven or nonwoven and formed from one or more of thermoplastic polymeric yarns, for example nylons, polyesters, PEEK (polyether ether ketone), EVA (ethylene vinyl acetate), silicones, PTFE (polytetrafluorinated ethylene), FEP (fluorinated ethylene propylene), PET/PBT (polyethylene terephthalate/polybutylene terephthalate, acetals, PA (polyacrylate), or PC (polycarbonate).

5. The device of claim 1, wherein the magnet is formed from ceramic or elemental material.

6. The device of claim 5, wherein the magnet is formed from Neodymium.

7. The device of claim 5, wherein the magnet is coated with a protective barrier material one or both sides.

8. The device of claim 7, wherein the magnet is coated with a protective barrier of an aramide material.

9. The device of claim 7, wherein said medical instrument is one or more of an Adson forceps, Debakey forceps, Brown forceps, Gerald forceps, Russian forceps, hemostat clamp, mosquito clamp, Kelly clamp, right angle clamp, Freer elevator, Army Navy retractor, metal suction tool, or manual clip applier.

10. The device of claim 7, wherein said device is carried by a vest worn by an operator underneath a surgical gown.

11. A method for an operator in a surgical theater to have access to a device carrying one or more medical instruments, which comprises the steps of:
(1) providing a device, which comprises providing a device comprising:
(a) a landing pad attachable to a surgical gown about the region between the abdomen and chest of a person wearing the surgical gown; and
(b) a magnet attached to the landing pad; and
(2) affixing one or more medical instruments to the magnet whereby the operator can access the on more medical instruments in the operating theater.

12. The method of claim 11, which additional comprises the step of coating the magnet coated a polymeric material on one or both sides.

13. The method of claim 1, which additional comprises the step of attaching more than one magnet to the landing pad.

14. The method of claim 11, which additional comprises the step of providing the landing pad comprising a woven or nonwoven material and formed from one or more of thermoplastic polymeric yarns, for example nylons, polyesters, PEEK (polyether ether ketone), EVA (ethylene vinyl acetate), silicones, PTFE (polytetrafluorinated ethylene), FEP (fluorinated ethylene propylene), PET/PBT (polyethylene terephthalate/polybutylene terephthalate, acetals, PA (polyacrylate), or PC (polycarbonate).

15. The method of claim 11, which additional comprises the step of forming the magnet from ceramic or an elemental material.

16. The method of claim 15, which additional comprises the step of forming the magnet from Neodymium.

17. The method of claim 15, which additional comprises the step of coating the magnet with a protective barrier material on one or both sides.

18. The method of claim 17, which additional comprises the step of coating the magnet with a protective barrier of an aramide material.

19. The method of claim 17, which additional comprises the step of affixing to the landing pad one or more of an Adson forceps, Debakey forceps, Brown forceps, Gerald forceps, Russian forceps, hemostat clamp, mosquito clamp, Kelly clamp, right angle clamp, Freer elevator, Army Navy retractor, metal suction tool, or manual clip applier.

20. The method of claim 17, which additional comprises the step of affixing said device to a vest worn by an operator underneath a surgical gown.
